(19) United States
(12) Patent Application Publication
(10) Pub. No.: US 2006/0129027 A1
(43) Pub. Date: Jun. 15, 2006

(54) Device for treating obesity

(76) Inventor: Antonio Catona, Pavia (IT)

Correspondence Address: WENDEROTH, LIND & PONACK, L.L.P., 2033 K STREET N. W., SUITE 800, WASHINGTON, DC, 20006-1021, US

(21) Appl. No.: 11/296,258

(22) Filed: Dec. 8, 2005

(30) Foreign Application Priority Data
Dec. 9, 2004 (EP) 04 029 186.6

(51) Int. Cl. A61F 2/00

(52) U.S. Cl. 600/37

(57) ABSTRACT

A device for treating obesity comprises a band part (1), which can be placed annularly about the stomach and includes a closure means (2, 3) for closing the gastric band after it has been placed annularly about the stomach, and an enveloping part (18) for enveloping and supporting a stomach region proximal with respect to the applied band part (1). The band part (1) and the enveloping part (18), secured in position on the band part (1) and laterally projecting from it, are implemented as a unit prefabricated by the manufacturer.

### BACKGROUND OF THE INVENTION

a) Field of the Invention

The invention relates to a device for treating obesity comprising a band part, which can be placed annularly about the stomach and which has closure means for closing the gastric band device after it has been placed annularly about the stomach, and an enveloping part for enveloping and supporting a stomach region proximal with respect to the applied band part.

b) Description of Related Prior Art

A gastric band device with a band part annularly placeable about the stomach and closure means is known for example from EP 0 702 529. The gastric band device comprises a band part to be placed about the stomach in the proximity of the inlet of the stomach, which band part has a longitudinally extending inner hollow chamber. At both ends of the band part are disposed first and second closure parts which form a closure means of the gastric band. To narrow the passage cross section of the gastric band opening of the annularly closed gastric band, the hollow chamber of the band part is filled with a filler medium, the quantity of the filler medium depending on the desired passage cross section. Filling the gastric band with the filler medium takes place through an injection port implanted under the skin of the patient and connected via tubules with the gastric band.

With implanted gastric band devices various complications may occur over time. One of these complications comprises that the region of the stomach in front of the gastric band, thus proximal to the gastric band, expands leading to disadvantageous effects for the patient, for example to a decrease of the effectiveness of the gastric band.

A device of the above described type is disclosed in U.S. Pat. No. 6,572,627 B2. The gastric band device comprises a band part and closure extensions projecting from it at both ends. U.S. Pat. No. 6,572,627 B2 further describes a perforated sheet. After the gastric band has been placed about the stomach, this sheet is wound about a section of the stomach lying proximal to the applied gastric band and fixed by suturing the overlapping portions. The sheet is moreover sutured to the gastric band. The enveloping part thus formed serves for limiting the expansion of this stomach section. Of disadvantage in this device is inter alia the complicated implantation. In order to define a correct value for the volume encompassed by the enveloping part, a balloon is introduced into the stomach and expanded to a suitable size before the stomach section is wrapped with the enveloping part.

### AIM AND SUMMARY OF THE INVENTION

The invention addresses the problem of providing an improved device of the above described type. This problem is resolved by a gastric band device comprising a band part to be placed annularly about the stomach with closure means for closing the gastric band after it has been placed annularly about the stomach, and an enveloping part for enveloping and supporting a stomach region located proximally with respect to the applied band part, the band part and the enveloping part, fixed in position on the band part and projecting laterally from it, are developed as a unit prefabricated by the manufacturer.

A device according to the invention in which the band part and the enveloping part fastened on it are implemented as a unit prefabricated by the manufacturer, consequently forms a gastric band with an integrated enveloping part. This prefabricated unit is implantable as a whole into the body of the patient. The enveloping part is preferably integrally connected with the band part, i.e. it is not removable without destroying the prefabricated unit.

Through the invention a simple and reliable implantation is achieved, the risk of surgical errors being reduced. A gastric band according to the invention can advantageously be applied laparoscopically.

The enveloping part can be adhered on the band part. An integral implementation of the enveloping part with the band part (or a portion hereof) is conceivable and possible.

The elastic enveloping part supports the stomach wall enveloped by it. For example, after the band part has been placed about the stomach and the closure means has been closed, the ends of the enveloping part now adjacent to one another, can be sutured with one another within the scope of the implantation of the gastric band, such that the enveloping part forms a type of circumferentially closed support collar for the enveloped region of the stomach wall and for the optionally also enveloped gastroesophageal transition zone. After the application of the band part about the stomach and the closing of the closure means, the ends of the enveloping part can also overlap and be sutured together in the overlapping portions.

In a further embodiment variant of the invention the enveloping part could be provided with closure parts, which, after the stomach region has been enveloped, can be closable whereby a circumferentially closed collar is formed by the enveloping part.

The enveloping part advantageously comprises a multiplicity of through-openings distributed over its extent, the sum of whose areas represents a major portion of the total area of the enveloping part. In an advantageous embodiment of the invention the enveloping part is implemented in the form of netting, i.e. it has intersecting strands in the form of a mesh, which are connected at the crossing points. To implement the enveloping part such that it is elastic, it is comprised of an elastic synthetic material.

The enveloping part preferably has at least one window opening, which, after the application of the gastric band, serves for the purpose of suturing stomach tissue, which is pulled over the band part (from the side of the stomach distal to the band part) to stomach tissue located under the window opening. Slipping of the gastric band in the course of time is thereby counteracted. This at least one window opening can be, for example, rectangular, square or circular. It is preferably at least large enough for a circle of 0.5 cm diameter to be inscribed in it.

Further advantages and details of the invention will be described in the following in conjunction with the embodiment example depicted in the enclosed drawing, based on which further tasks of the invention are evident.

### BRIEF DESCRIPTION OF THE DRAWINGS

In the drawing depict:

FIG. 1 a side view of an embodiment example of a gastric band according to the invention in the closed state,

FIG. 2 and 3 perspective view of the gastric band of FIG. 1 from different viewing angles,

FIG. 4 a section along line A-A of FIG. 1,

FIG. 5 and 6 perspective representations of the gastric band of FIG. 1 from different viewing angles, in the open state of the closure means,

FIG. 7 a schematic representation of the gastric band placed about the stomach with connected injection port,

FIG. 8 in elevation a slightly modified embodiment of an enveloping part laid out flat,

FIG. 9 a schematic illustration of the two end regions (in opposing position) of a further embodiment of an enveloping part.

### DESCRIPTION OF THE PREFERRED EMBODIMENT EXAMPLE

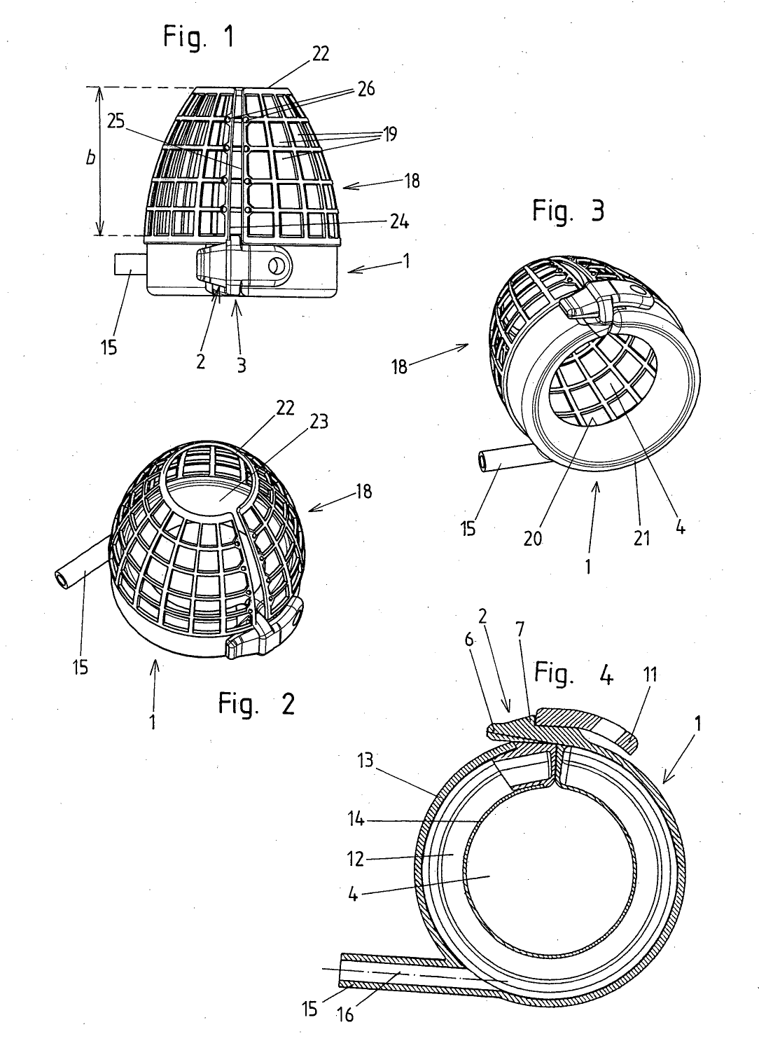

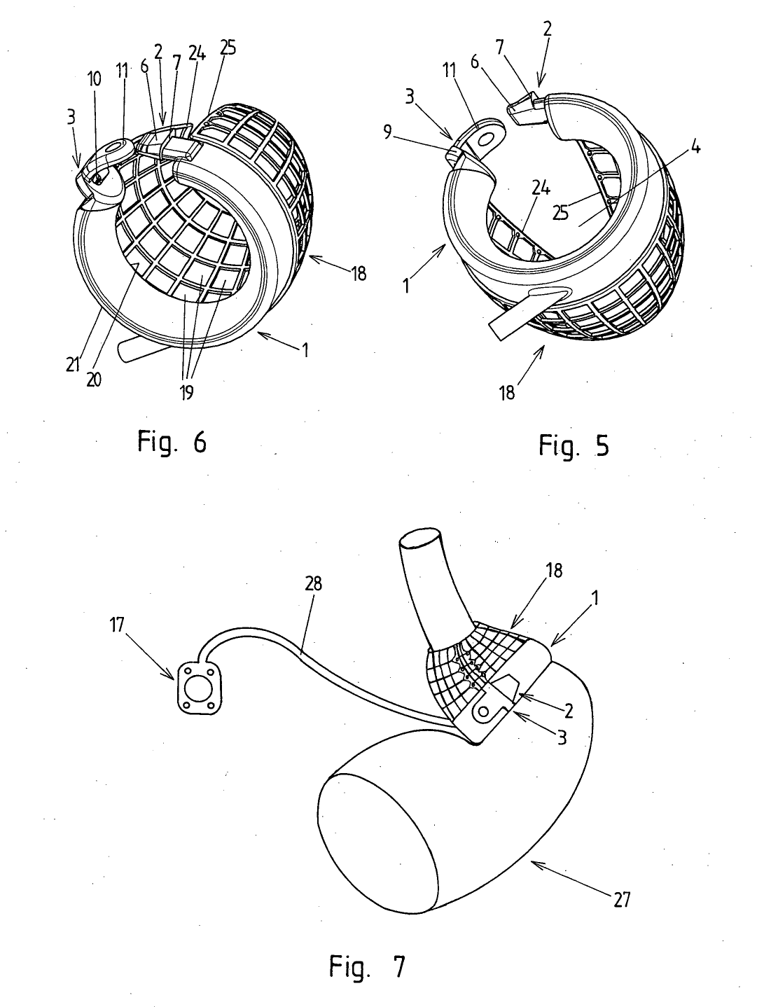

The embodiment example depicted in FIG. 1 to 6 of a gastric band according to the invention comprises a band part 1, at whose two ends are disposed first and second closure parts 2, 3, which represent a closure means for connecting the two end regions of the band part 1. When the closure means (FIG. 1 to 4) is closed, a circumferentially closed ring is formed which encloses gastric band opening 4. The first closure part 2 has an extension 6 projecting beyond the frontal end 5 of the band part 1 and provided with a latch projection 7. The second closure part 3 is formed by a web 9, projecting radially outwardly with respect to the band part 1 in the proximity of the other frontal end 8, which has an insertion slot 10 for the extension 6. To facilitate the closing of the closure, on web 9 further a pull tab 11 is disposed. To close the closure means, the extension 6 is pulled through the insertion slot 10 until the latch projection 7 has passed through the insertion slot 10 and reaches behind the edge of the insertion slot 10.

The extension 6 with its latch projection 7 and web 9 are preferably implemented of an elastomer, in particular silicon, with such hardness and further with such cooperating geometry, that the closure means opens under a tearing pull exceeding a specified limit value. Thereby a safety closure can be provided through which an impermissibly high pressure in the stomach can be avoided.

The closure parts 2, 3 disposed at both ends of the band part may be disposed on plugs which project into the ends of the band part 1 and which close off the hollow chamber 12 of the band part 1. The plugs may, for example, be adhered in. The implementation of the closure means, or portions thereof, as a material unit with the band part 1 is conceivable and possible. The band part 1 may be comprised of an elastomer, preferably silicon.

In the depicted embodiment example, the band part 1 is formed by a tube, closed in the region of its two ends, in which extends the hollow chamber 12, which thus, in the depicted embodiment example, extends substantially over the entire length of band part 1. The backing section 13 (which is the wall delimiting the hollow chamber 12 against the outside) of band part 1 has a lesser elastic extensibility than the inner section 14. This can be achieved, for example, by implementing the backing section such that it is thicker and/or harder. In addition, or instead, the backing section 13 can also be reinforced by a reinforcing layer, which, for example, is embedded in the material of the backing section or be adhered on its side facing away from the hollow chamber 12 and extends over the length of the band part 1. This reinforcing layer can herein comprise filaments continuous in the longitudinal direction and advantageously also in the transverse direction over its entire extent and can, in particular, also be implemented as a rectangular weave for example of a synthetic material. Due to the less elastic or non-elastic implementation of the backing section, its expansion during the filling of the band part 1 with a filler medium is decreased or prevented and the tensile stability of the gastric band is ensured.

The gastric band device has further a connection tubule 15, whose inner channel 16 terminates in the hollow chamber 12. The connection tubule 15 serves for connecting an injection port 17 (cf. FIG. 7) to the gastric band.

The gastric band device comprises furthermore an elastic enveloping part 18 laterally projecting from band part 1. For example, the projecting width b of the enveloping part is in the range of 6 to 10 cm measured in the direction perpendicularly to the longitudinal extent of the band part 1. The enveloping part 18 is a flat structure, i.e. its thickness, which is less than 5 mm and preferably less than 2 mm, is substantially less than its width or length. In the depicted embodiment example the enveloping part 18 extends over the length of the band part 1. In principle, it would also be conceivable and possible that it projects beyond the frontal ends of band part 1, such that, in the closed state of the gastric band, overlapping portions of the enveloping part are formed.

The enveloping part 18 has a multiplicity of through-openings 19 distributed over its extent. The enveloping part is preferably, as shown, implemented in the form of a netting (mesh-like). For the elastic support of the stomach wall the enveloping part is comprised of an elastomer, for example of silicon, whose hardness can be, in particular, in the range between 30 and 60 Shore A. In order to counteract migration of the enveloping part into the stomach tissue, the enveloping part can be coated with titanium at least on the side facing the stomach wall, or all around, when the gastric band device is applied. The coating thickness can herein be very narrow and be, for example, only a few atomic layers thick.

Although it is preferred that the enveloping part 18 laterally projects only beyond one of the two longitudinal edges 20, 21 of band part 1, it would also be conceivable and possible to provide an enveloping part projecting on both sides of band part 1 (implemented in one-piece or comprised of two separate parts, each fastened on the band part 1), for example to counteract more strongly migration of the gastric band device into the stomach tissue.

In the depicted embodiment example the enveloping part 18 is attached, for example adhered, in the region of the longitudinal edge 20 of band part 1.

FIG. 1 to 6 depict the gastric band device in the state in which it is not applied around the stomach. In the closed state of the closure means 2, 3 the enveloping part 18 here has a cupola- or dome-form shape, i.e. when the gastric band device is viewed from the side (FIG. 1), the side edges of the enveloping part have an arcuate course, which extends up to the free longitudinal edge 22 of the enveloping part remote from band part 1, the distance from the longitudinal center axis of the gastric band decreasing (no external force is effective here). The free longitudinal edge 22 of enveloping part 18 encompasses an opening 23, which preferably has a diameter in the range of 2 to 3 cm, a value of approximately 2.5 cm being especially preferred.

In the case of the depicted embodiment example the frontal ends 24, 25 of enveloping part 18 are adjacent to one another if closure means 2, 3 is closed. After the gastric band device is placed about the stomach, they can be sutured to one another and, in the depicted example, suture loops 26 are provided.

FIG. 7 shows schematically the gastric band device after it has been placed about the stomach 27. Band part 1 has been placed about a proximal region of the stomach 27 and the closure means 2, 3 has been closed. The two frontal ends 24, 25 of the enveloping part 18 have been sutured to one another, such that the enveloping part 18, in the manner of a collar, encompasses at least the region of the stomach 27 which is proximal (directed toward the cardia) with respect to band part 1. The gastroesophageal transition zone (in the interior of which is located the cardiac sphincter) can also be encompassed by the enveloping part 12. The elasticity in combination with the geometric form of the enveloping part 18 is here so selected that it does not fit too tightly around the stomach (otherwise the risk of migration would be encountered)) on the other hand, however, elastically supports the stomach wall to an adequate degree.

Subsequently, by means of a connection tubule 28, to the gastric band device an injection port 17 is connected which is implanted under the skin of the patient. The size of the gastric band opening 4 can subsequently be adjusted by injecting filler medium into the injection port 17 through a needle.

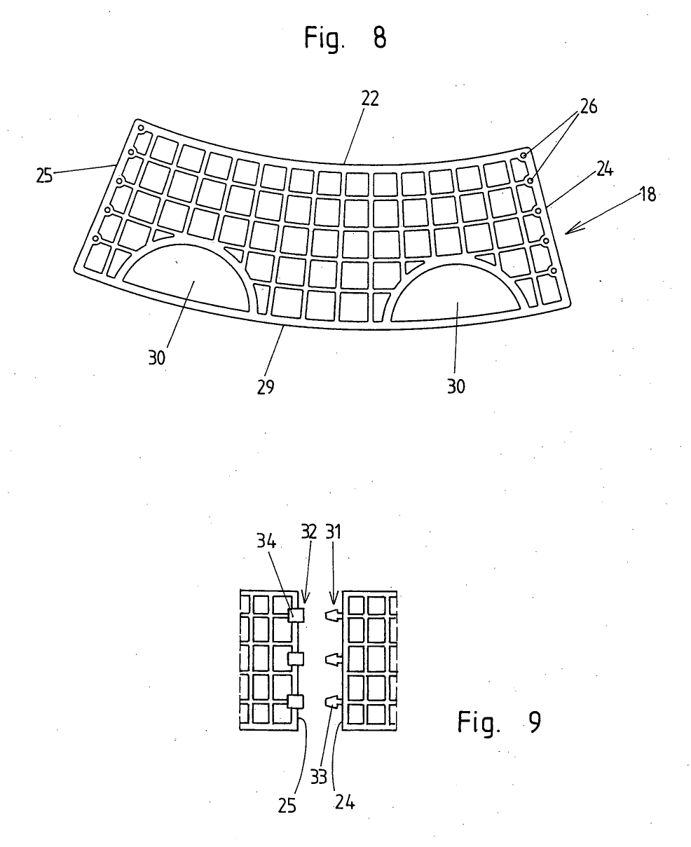

A further embodiment example of an enveloping part 18 is depicted in FIG. 8. The enveloping part 18 is adhered to the band part 1 in the region of its longitudinal edge 29. In the proximity of the longitudinal edge 29 two window openings 30 are formed. After the band part 1 has been applied about the stomach and the two frontal ends 24, 25 of the enveloping part 18 have been sutured, stomach tissue can be pulled over band part 1 and through the window opening 30 be sutured to stomach tissue located beneath the enveloping part 18 in order to counteract the slipping of the gastric band occurring over time.

Instead of suturing the frontal ends 24, 25 or overlapping portions of the enveloping part 16, it would also be conceivable and possible to provide the enveloping part 18 in the region of its frontal ends or its overlapping portions with a closure means 31, 32, such as is schematically depicted in FIG. 9 by example. In the embodiment example schematically depicted in FIG. 9 at one frontal end 24 of the enveloping part are disposed extensions 33 with latch projections, which can be slid into insertion slots located in webs 34 on the other frontal end 25, with the latch projections of the extensions 33, after they have been inserted, reaching behind the edges of the insertion slots in webs 34. Other means of closure, for example by means of hook-like elements, would also be conceivable and possible.

Different modifications of the depicted embodiment example are conceivable and possible without going beyond the scope of the invention. For example, the closure means for the band part could also be formed differently from that shown. Conceivable and possible would be, for example, to implement the enveloping part as a punched film instead of in the form of a netting or mesh. It would in principle also be conceivable and possible to implement the band part without an inner hollow chamber. It could also be provided to adjust the diameter of the gastric band opening by means of a cylinder-piston unit, the piston rod of the piston being fastened in the proximity of the one end of the band part 1 and the cylinder in the proximity of the other end of band part 1 and at least one of these parts (piston rod and cylinder) being fastened (for example by means of a snap closure) only after the band part has been placed about the stomach. The position of the piston with respect to the cylinder could, again, be adjusted by means of a connected injection port.

It would also be conceivable and possible to coat the first closure part 2 and/or the second closure part 3 with titanium in order to prevent adhesions in this region. Later removal of the gastric band device is thereby facilitated. Coating of the band part 1 with titanium, in particular on its side facing the stomach, is also conceivable and possible in order to counteract migration of the band part into the stomach tissue. Titanium coatings can be provided wherever adhesions are undesirable. The coating thickness can again be very narrow and be, for example, only a few atomic layers thick.

As emerges from the above description, the scope of the invention is not limited to the depicted embodiment examples, but rather, should be determined with reference to the attached claims together with their full scope of possible equivalents. While the above description and the drawings represent the invention, it is apparent to a person of skill in the art, that various modifications can be carried out therein without leaving the true spirit and scope of the invention.

### LEGEND TO THE REFERENCE NUMBERS

1 Band part
2 First closure part
3 Second closure part
4 Gastric band opening
6 Extension
7 Latch projection
9 Web
10 Insertion slot
11 Pull tab
12 Hollow chamber
13 Backing section
14 Inner section
15 Connection tubule
16 Inner channel
17 Enveloping part
19 Through-opening
20 Longitudinal edge
21 Longitudinal edge
22 Free longitudinal edge
23 Opening
24 Frontal edge
25 Frontal edge
26 Suture loop
27 Stomach
28 Connection tubule
29 Longitudinal edge
30 Window opening
32 Closure part
33 Closure part
33 Extension
34 Web

1. A device for the treatment of obesity comprising a band part (1), which can be placed annularly about the stomach and which includes a closure means (2, 3) for closing the gastric band after it has been placed annularly about the stomach, and an enveloping part (18) for enveloping and supporting a stomach region proximal with respect to the applied band part (1), wherein the band part (1) and the enveloping part (18), secured in position on the band part (1) and laterally projecting from it, are implemented as a unit prefabricated by the manufacturer.

2. The device as claimed in claim 1, wherein the enveloping part (18) comprises a multiplicity of through-openings (19) distributed over its extent.

3. The device as claimed in claim 2, wherein the sum of the areas of the through-openings (19) represents the major portion of the total area of the enveloping part (18).

4. The device as claimed in claim 2, wherein the enveloping part (18) is implemented in the form of netting.

5. The device as claimed in claim 1, wherein the enveloping part (18) is adhered on the band part (1).

6. The device as claimed in claim 5, wherein the enveloping part (18) is adhered in the region of one of the longitudinal edges (20, 21) of the band part (1).

7. The device as claimed in claim 1, wherein the two frontal ends (24, 25) of the enveloping part (18), after the gastric band device has been placed about the stomach, are adjacent to one another or portions of the enveloping part (18) adjoining the two frontal ends (24, 25) overlap and, for the formation of a circumferentially closed support collar, the frontal ends (24, 25) adjacent to one another or the overlapping portions adjoining the two frontal ends (24, 25) can be fastened to one another.

8. The device as claimed in claim 7, wherein the frontal ends (24, 25) or the overlapping portions of the enveloping part (18) adjoining the frontal ends (24, 25) can be sutured to one another for their mutual fastening.

9. The device as claimed in claim 8, wherein suture loops (26) are provided for the suturing.

10. The device as claimed in claim 7, wherein a closure means is provided for the mutual fastening of the frontal ends (24, 25) or of the overlapping portions of the enveloping part (18) adjoining the frontal ends (24, 25).

11. The device as claimed in claim 10, wherein the closure means is formed by closure parts (31, 32) which can be latched with one another.

12. The device as claimed in claim 1, wherein the enveloping part (18) extends at least over the length of the band part (1).

13. The device as claimed in claim 1, wherein the width (b) of the enveloping part (18) projecting laterally from the band part (1) is at least 2 cm measured in the direction perpendicularly to the longitudinal extent of the band part (1),

14. The device as claimed in claim 13, wherein the width (b) of the enveloping part (18) projecting laterally from the band part (1) is at most 6 cm measured in the direction perpendicularly to the longitudinal extent of the band part (1).

15. The device as claimed in claim 1, wherein the enveloping part (18) is comprised of an elastic synthetic material.

16. The device as claimed in claim 15, wherein the enveloping part (18) is comprised of silicon.

17. The device as claimed in claim 1, wherein the hardness of the enveloping part (18) is in the range between 30 and 60 Shore A.

18. The device as claimed in claim 1, wherein the enveloping part (18) includes at least one window opening (30) for suturing the stomach tissue pulled over the applied band part (1) to the stomach tissue beneath the window opening (30).

19. The device as claimed in claim 1, wherein the enveloping part (18) is coated with titanium at least on the side facing the stomach wall after the gastric band has been applied.

20. The device as claimed in claim 1, wherein, after the gastric band has been applied, the enveloping part (18) encompasses at least the region of the stomach (27) proximal to the enveloping part (18). [SIC: the band part (1)]

21. The device as claimed in claim 20, wherein, after the gastric band has been applied, the enveloping part (18) also encompasses the gastroesophageal transition zone.

22. The device as claimed in claim 1, wherein the band part (1) comprises at least one hollow chamber (12) which [extends at least over a portion of the band part (1) and] can be fillled with a filler medium.

23. The device as claimed in claim 22, wherein the hollow chamber (12) fillable with a filler medium extends at least over a major portion of the length of the band part (1), and wherein when filling the hollow chamber (12) an inner section (14) of the band part (1) delimting the gastric band opening (4) expands toward the axis of the gastric band opening (4) decreasing the gastric band opening (4).

24. The device as claimed in claim 1, wherein, with the closure means (2, 3) closed, the enveloping part (18) has a cupola- or dome-form shape without an external force acting on the enveloping part (18).

25. Method for treating obesity, wherein a band part (1) comprising a closure means (2, 3) and on which is disposed an enveloping part (18) for enveloping and supporting a stomach region proximal with respect to the applied band part (1), is placed annularly about the stomach and the closure means (2, 3) of the band part (1) is closed to form an annular gastric band device, and the two frontal ends (24, 25) of the enveloping part (18), which are adjacent to one another, or the overlapping portions of the enveloping part (18) adjoining the two frontal ends (24, 25), are fastened with one another to form a circumferentially closed support collar.
